(19) United States
(12) Patent Application Publication
(10) Pub. No.: US 2019/0185250 A1
(43) Pub. Date: Jun. 20, 2019

(54) A PRESSURISED CONTAINER

(71) Applicant: Kind Consumer Limited, London (GB)

(72) Inventors: Khine Zaw NYEIN, London (GB); Stephen Wynford MORRIS, Sussex (GB)

(73) Assignee: Kind Consumer Limited, London (GB)

(21) Appl. No.: 16/311,476

(22) PCT Filed: Jun. 19, 2017
(86) PCT No.: PCT/GB2017/051787

§ 371 Date: Dec. 19, 2018

(30) Foreign Application Priority Data
Jun. 20, 2016 (GB) 1610702.1

(51) Int. Cl. B65D 83/48; A61M 11/02; A61M 15/00; A61M 16/20

(52) U.S. Cl. CPC B65D 83/48; A61M 16/201; A61M 15/009; A61M 11/02

(57) ABSTRACT

A pressurised container containing a liquid composition to be dispensed and a propellant to pressurise the container. The container has a non-metred outlet valve at its lower end such that it is in communication with liquid in the container when the container is oriented with the valve lowermost. The valve comprises a valve stem having an outlet towards the bottom end communicating with an inlet as a lateral port through the side of the stem, the lateral port being positioned such that it is moveable between a first position out of communication with the liquid and a second position in communication with the liquids. A resilient element biases the valve element to the first position. A sealing plate which surrounds the valve stem and has a first seal and a second seal. An end cap is fitted over the sealing plate.

The present invention relates to a pressurised container. In particular, the invention is concerned with a simplified design of the end of the container including the valve in order to provide a non-metred flow from the pressurised container.

There are two methods of dispensing aerosol product through a valve system known in pharmaceutical industry, namely metering valves to deliver aerosol products to the users in a finite volume or quantity, and non-metering valve system to dispense a similar product but in a continuous or unrestricted flow either directly to the users or through intermediary units or devices.

Metering valves are prevalent in pharmaceutical industry as it is essential for them to deliver a highly accurate dose to the users or patients. Their delivery method is typically direct to the users and invasive in nature, and hence the importance of controlling the treatment doses for users' health and safety. One very common example product is a pressurised metered dose inhaler (PMDI) for treating asthma which can deliver a defined quantity of pharmaceutical aerosol to users for their treatment or inhalation use.

Non-metering or continuous flow valves are found in a wide variety of industrial applications, also including pharmaceutical applications. Pharmaceutical valves of this type (non-metering) are most commonly found in topical applications where the accuracy level of the doses are not as stringent.

An example of such a non-metred valve is disclosed in WO 2014/155089. This is based on the design of a metred valve. However, the shape of the valve stem is changed such that it does not seal with the innermost seal which is provided in a metred valve in order to create the metering chamber. The valve in this document consists of numerous components as it is to provide the metering chamber. There is also a relatively large cage/housing provided in order to retain the metering chamber and the spring which biases the valve element.

The present invention is designed to significantly simplify such a design.

According to the present invention there is provided a pressurised container according to claim 1.

Because the sealing plate provides the seal both with the container body and the valve stem as well as supporting the resilient element, the structure of the bottom end of the container is greatly simplified as compared to the prior art.

The first and second seals may be formed separately and attached to the sealing plate but are preferably formed integrally with the sealing plate.

The resilient element may be formed integrally with the sealing plate. For example, there may be a resilient portion at the inner edge of the sealing plate which extends away from the plate to create a resilient sleeve providing the resilient element for the valve stem. This may be the same component as the first seal. Forming the resilient element integrally with the sealing plate further reduces the number of components and therefore simplifies the assembly. However, the resilient element may equally be a separate element such as a coiled spring or a resilient sleeve thereby allowing the container to be manufactured using standard components. Preferably, the sealing plate comprises a portion of greater rigidity than the first and second seals in the vicinity of the resilient element.

The resilient element may be positioned inside the pressurised space. However, it is preferably positioned outside of the pressurised space. This means that the only part of the valve assembly within the pressurised part of the container is the inlet end of the valve stem thereby significantly reducing the amount of space within the pressurised container occupied by the valve assembly. This is significant in an application where space is at a premium.

Preferably the inner surface of the plate defines the lower boundary of the container and the lateral bore aligns with this inner surface in the second position. This allows all of the liquid within the container to be dispensed as compared to the valve of WO 2014/155089 where there are a number of regions in which the liquid can become inaccessible. Again this allows for a space saving as a smaller container can be used to dispense the same amount of liquid, as well as reducing the expense of wasting a certain amount of the liquid composition.

Examples of a pressurised container in accordance with the present invention will now be described with reference to the accompanying drawings, in which:

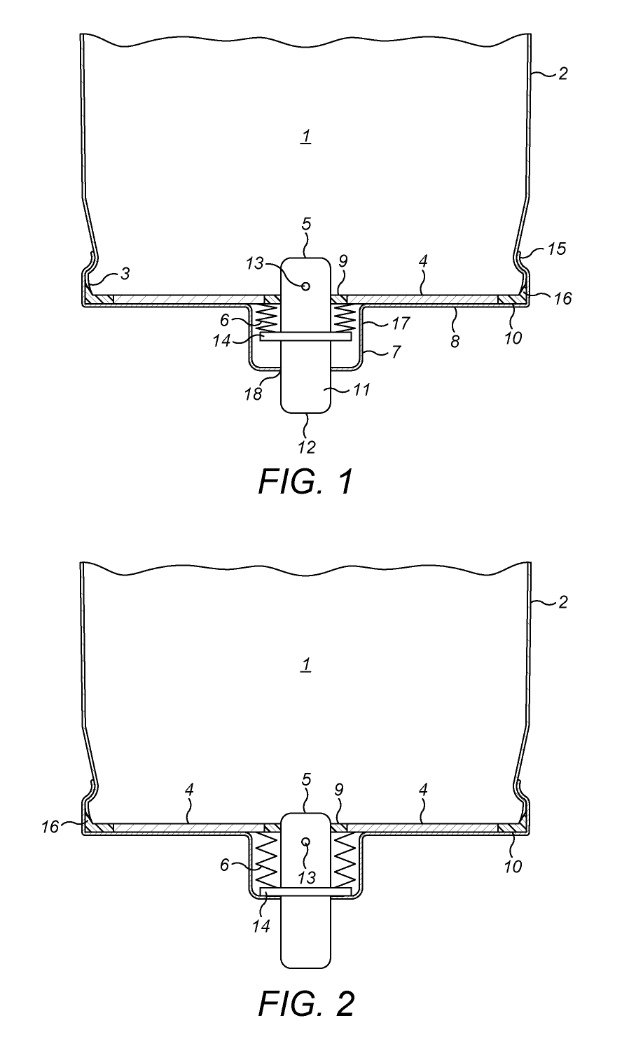

FIG. 1 is a cross-sectional view through a first example of a bottom end of a container with the valve open;

FIG. 2 is a view similar to FIG. 1 with the valve closed;

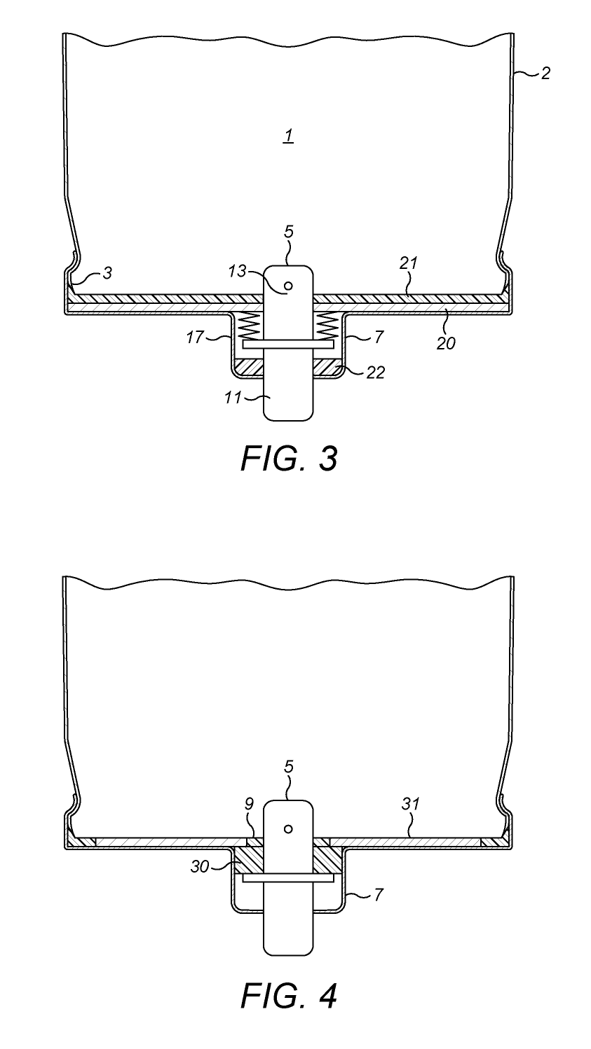

FIG. 3 is a view similar to FIG. 1 of a second example; and

FIG. 4 is a view similar to FIG. 1 of a third example.

The pressurised container 1 will contain a liquid and a propellant in order to pressurise the container. The liquid may be any substance which is dispensed in an aerosol form. One specific example of a container is as a refill device for a simulated cigarette in which case the formulation will include nicotine. The container may be designed to dispense the material into the surrounding environment, or may be designed to refill a device such as a simulated cigarette.

The container is explicitly designed for use in an inverted configuration meaning that it can only be used in the orientation shown in the Figures with the outlet valve lowermost. This means that the liquid within the container 1 will be present in the bottom of the container and will be pressurised by the propellant. Because the container is to be used in an inverted configuration, it does not require a dip tube.

The container comprises a container body 2 with a lower peripheral lip 3. The container body 2 may be of a conventional design. The novelty resides in the design of the lower part of the container including the valve assembly.

As shown in the drawings, the lower part of the assembly consists of just four components, namely a sealing plate 4, a valve stem 5, a resilient element 6 and an end cap 7 known as a ferrule.

These components will be described in more detail below.

The sealing plate 4 is the component which both seals the container 1 and also seals with the valve stem 5. As shown in the Figures, the sealing plate is annular and has a planar configuration. It does not necessarily require the planar configuration as it could, for example, have a shallow frustoconical or other non-planar configuration. This could be helpful in pooling the liquid in the vicinity of the stem. The sealing plate has a rigid intermediate portion 8 which provides sufficient rigidity to withstand the pressure in the container 1 as well as being able to support the resilient element 6.

At its inner periphery, the sealing plate 4 has a sealing element 9 and similarly has a second sealing element 10 at its outer periphery. The sealing elements 9, 10 may, for example be a rubber material. The sealing plate 4 could be formed, for example, as a metal or plastic washer which is dipped in rubber to form the sealing elements 9, 10. Alternatively the plate could be a plastic/rubber co-moulding. Another possibility is for it to be made of a resilient material such as rubber with reinforcing elements which provide additional rigidity to the intermediate portion 8.

The valve stem 5 has an axial bore 11 extending to an outlet 12. The outlet 12 is shown at the axial end of the valve stem 5, but could be elsewhere towards the lower end of the stem 5. The inlet to the valve stem 5 is provided by a lateral bore 13 which, in the valve open position of FIG. 1 is positioned immediately above the upper (i.e. inner) surface of the sealing plate 4.

As can be appreciated from FIG. 1, the space within the container 1 occupied by the valve assembly is minimal. Also, as can be seen in FIG. 1, all of the liquid in the container 1 can be dispensed through the valve stem 5 as there is no dead space in which any liquid will be trapped.

The valve stem 5 has an outwardly projecting flange 14 which engages with a resilient element 6 which, in the first example, is a helical spring. This bears against the plate 4 to provide a biasing force on the valve stem.

In order to complete the assembly, the sealing plate 4 is temporarily retained in position in the end cap 7 by a number of circumferentially spaced steps 16 before the end cap 7 is fitted into place on the container body 2.

The cap is crimped at its outer periphery 15 to the open end of the container body 2. The crimping force causes the lip 3 of the container body 2 to seal against the outer sealing element 10. The cap 7 has a recessed central portion 17 with a central opening 18. This recessed portion 17 fits over the valve stem 5 which protrudes through the opening 18 while the portion of the cap which surrounds the opening 18 serves to retain the valve stem 5 by engaging with a flange 14 as shown in FIG. 2.

To open the valve, the valve stem 5 is pushed upwardly from the position shown in FIG. 2 against the biasing force of the resilient element 6. This causes the lateral bore 13 to pass beyond the first sealing element 9 into the position shown in FIG. 1. In this position, the lateral bore 13 is in communication with liquid in the container 1 which is forced by the pressure along the lateral bore 13 and axial bore 11 and out of the outlet 12. The flow is unmetered so will continue until the inward pressure on the valve stem 5 is removed, whereupon the resilient element 6 will close the valve by biasing it to the downward position shown in FIG. 2.

A second example of a container is shown in FIG. 3. Here, the sealing plate 4 has a different configuration. Rather than having sealing elements exclusively at the periphery, it is provided as a two layer structure with a rigid layer 20 and a sealing layer 21. This sealing plate 4 otherwise functions in a similar manner to the first example in that the sealing layer 21 seals with the lip 3 of the container 2 towards its outer periphery and at its inner periphery seals with a valve stem 5. The rigid layer 20 supports the resilient element 6.

A second modification shown in FIG. 3 is the presence of an anti-leak seal 22. It is possible that some material which enters the lateral bore 13 and axial bore 11 will be retained in the valve stem 5 after the valve stem 5 moves to the closed position. This is only likely to happen with viscous liquids as the pressure is otherwise sufficient to force all of the liquid out of the valve stem 5. However, if any such liquid is trapped, it can enter the recess 17, but is prevented from leaking into the environment by the anti-leak seal 22.

The example shown in FIG. 4 is the same in all respects as the first example except that the resilient element 6 which is a helical spring in the first example has now been replaced with a resilient sleeve 30. This may be formed integrally with the inner sealing element 9. In FIG. 4, the end assembly therefore consists only of three components, namely the valve stem 5, the end cap 7 and the modified sealing plate 31.

1. A pressurised container containing a liquid composition to be dispensed and a propellant to pressurise the container,
the container having a non-metred outlet valve at its lower end such that it is in communication with liquid in the container when the container is oriented with the valve lowermost;
the valve comprising a valve stem having an outlet towards the bottom end communicating with an inlet as a lateral port through the side of the stem, the lateral port being positioned such that it is moveable between a first position out of communication with the liquid and a second position in communication with the liquid;
a resilient element biasing the valve element to the first position;
a sealing plate which surrounds the valve stem and has a first seal at its inner edge which seals against the valve stem, and a second seal towards its outer periphery to seal against a container body to create a sealed enclosure, the sealing plate further being positioned to support the resilient element; and
an end cap fitting over the sealing plate to attach the sealing plate to the container body, the end cap also being configured to retain the valve stem in place.

2. A container according to claim 1, wherein the first and second seals are formed integrally with the sealing plate.

3. A container according to claim 1, wherein the resilient element is formed integrally with the sealing plate.

4. A container according to claim 3, wherein the resilient element is a resilient portion at the inner edge of the sealing plate which extends away from the plate to create a resilient sleeve providing the resilient element for the valve stem.

5. A container according to claim 1, wherein the resilient element is positioned outside of the pressurised space.

6. A container according to claim 1, wherein the inner surface of the plate defines the lower boundary of the container and the lateral bore aligns with this inner surface in the second position.

7. A container according to claim 1 wherein the sealing plate further comprises a portion of greater rigidity than the first and second seals in the vicinity of the resilient element.

8. A container according to claim 1, wherein the only part of the valve within the pressurised part of the container is the inlet end of the valve stem.

9. A container according to claim 1, wherein there are no regions in the lower end of the container in which liquid can be trapped, when the container is oriented with the valve lowermost.
